(19) United States
(12) Patent Application Publication
(10) Pub. No.: US 2004/0029186 A1
(43) Pub. Date: Feb. 12, 2004

(54) Methods

(76) Inventors: Duncan Haworth, Macclesfield (GB); Lesley Margaret McCormick, Macclesfield (GB)

Correspondence Address: Janis K Fraser, Fish & Richardson, 225 Franklin Street, Boston, MA, 02110-2804, US

(21) Appl. No.: 10/312,481

(22) PCT Filed: Jun. 26, 2001
(86) PCT No.: PCT/GB01/02838

(30) Foreign Application Priority Data
Jun. 30, 2000 (GB) 0015923.6

(51) Int. Cl. G01N 33/53; G01N 33/567

(52) U.S. Cl. 435/7.2

(57) ABSTRACT

This invention relates to methods for measuring the binding interaction between a leukocyte adhesion molecule and a vascular endothelial ligand in whole blood ex vivo. In a preferred embodiment of the invention, the leukocyte adhesion molecule is the integrin α4β1, also known as Very Late Antigen-4 (VLA-4) or CD49d/CD29, and the vascular endothelial ligand is Vascular Cell Adhesion Molecule-1 (VCAM-1). The methods are especially useful for measuring the effects of compounds which modulate the binding interaction between the adhesion molecule and its ligand.

This invention relates to methods for measuring the binding interaction between a leukocyte adhesion molecule and a vascular endothelial ligand in whole blood ex vivo. In a preferred embodiment of the invention, the leukocyte adhesion molecule is the integrin α₄β₁, also known as Very Late Antigen-4 (VLA-4) or CD49d/CD29, and the vascular endothelial ligand is Vascular Cell Adhesion Molecule-1 (VCAM-1). A particular advantage of the methods of the invention is that the binding interaction between the leukocyte adhesion molecule and its vascular endothelial ligand can be measured in whole blood. The methods are especially useful for measuring the effects of compounds which modulate the binding interaction between the adhesion molecule and its ligand. Such compounds may have utility in the treatment of inflammatory diseases.

α₄β₁ is a member of the integrin family of heterodimeric cell surface receptors that are involved in the adhesion of cells to other cells or to extracellular matrix. The interactions between integrins and their protein ligands are fundamental for maintaining cell function, for example by tethering cells at a particular location, facilitating cell migration, or providing survival signals to cells from their environment. Ligands recognised by integrins include extracellular matrix proteins, such as collagen and fibronectin; plasma proteins, such as fibrinogen; and cell surface molecules, such as transmembrane proteins of the immunoglobulin superfamily and cell-bound complement. Integrins are composed of noncovalently associated glycoprotein subunits (α and β). There are at least 16 different human integrin α subunits and at least 8 different β subunits and each β subunit can form a heterodimer with one or more α subunits. The specificity of the interaction between integrin and ligand is governed by the α and β subunit composition.

α₄β₁ is expressed on numerous hematopoietic cells, including hematopoietic precursors, peripheral and cytotoxic T lymphocytes, B lymphocytes, monocytes, thymocytes and eosinophils, and established cell lines. α₄β₁ has two main ligands, Vascular Cell Adhesion Molecule-1 (VCAM-1), also known as CD106, an immunoglobulin superfamily member expressed on the surface of activated vascular endothelial cells and a variety of other cells including dendritic cells, macrophages and fibroblasts, and an isoform of fibronectin containing the alternatively spliced type III connecting segment (CS-1 fibronectin). The α₄ subunit also forms a heterodimer with the β₇ subunit. α₄β₇ also recognises VCAM-1 and CS-1 fibronectin as ligands but will preferentially bind to Mucosal Addressin Cell Adhesion Molecule-1 (MAdCAM-1), another immunoglobulin superfamily member expressed on vascular endothelial cells, mainly in the small intestine and to a lesser extent the colon and spleen. α₄β₇ is expressed on lymphocytes that preferentially home to gastrointestinal mucosa and gut-associated lymphoid tissue and may have a role in maintaining mucosal immunity.

The activation and extravasation of blood leukocytes plays a major role in the development and progression of inflammatory diseases. Cell adhesion to the vascular endothelium is required before cells migrate from the blood into inflamed tissue and is mediated by specific interactions between cell adhesion molecules on the surface of vascular endothelial cells and circulating leukocytes.

α₄β₁ is believed to have an important role in the recruitment of lymphocytes, monocytes and eosinophils during inflammation. Expression of the α₄β₁ ligand VCAM-1 is upregulated on endothelial cells in vitro by inflammatory cytokines. VCAM-1 expression is also upregulated in human inflammatory diseases such as rheumatoid arthritis, multiple sclerosis, allergic asthma and atherosclerosis while CS-1 fibronectin expression is upregulated in rheumatoid arthritis. Monoclonal antibodies directed against the α₄ integrin subunit and small molecule α₄β₁ inhibitors have been shown to be effective in a number of animal models of human inflammatory diseases including multiple sclerosis, rheumatoid arthritis, allergic asthma, contact dermatitis, transplant rejection, insulin-dependent diabetes, inflammatory bowel disease, and glomerulonephritis.

Compounds which inhibit the binding interaction between α₄β₁ and VCAM-1 are expected to be effective treatments for a number of human inflammatory diseases or conditions, including asthma, rheumatoid arthritis, multiple sclerosis, transplant rejection, atherosclerosis, contact dermatitis, insulin-dependent diabetes, inflammatory bowel disease and glomerulonephritis. Examples of compounds which inhibit the binding interaction between α₄β₁ and VCAM-1 are disclosed in WO 96/20216, WO 97/02289, WO 97/49731, WO 99/24398, WO 00/05223 and WO 00/05224, AstraZeneca.

The development of α₄β₁/VCAM-1 inhibitors would be facilitated by the ability to test their activity in whole blood samples after dosing to humans or animals as this would provide a good indicator of their therapeutic effect, i.e. the inhibition of α₄β₁-expressing leukocyte recruitment from the blood following interaction with ligands expressed on vascular endothelial cells. However, no such whole blood assay has yet been reported.

Currently available methods which have been used to search for α₄β₁/VCAM-1 inhibitors involve screening in vitro either for inhibition of the interaction between purified α₄ β₁ and ligand proteins or for inhibition of the interaction of α₄β₁-expressing cells and ligand. For example, Vanderslice et al (J.Immunol. 1977, 158, 1710-1718) measured inhibition of binding of fluorescently-labelled HL-60 and Ramos cell lines to VCAM-IgG-conjugated beads. Jackson et al (J. Med Chem. 1997, 40, 3359-3368) measured inhibition of binding of purified α₄β₁ to VCAM-1 bound on a 96-well plate using an enzyme-linked immunosorbant assay (ELISA) format and inhibition of Ramos cell line adhesion to 96-well plates coated with a VCAM-1-IgG chimera. Lin et al (J. Med. Chem. 1999, 42, 920-934) measured inhibition of binding of VCAM-Ig conjugated with alkaline phosphatase. Haworth et al (Brit. J. Pharmacol. 1999, 126, 1751-1760) used inhibition of MOLT-4 cell line adhesion to human plasma fibronectin coated on 96-well plates to identify α₄β₁ inhibitors.

Similar techniques can be used to measure the effects of compounds in vitro on the interaction of isolated, α₄β₁ expressing, blood leukocytes with α₄β₁ ligands. However, these methods are unsuitable for measuring the ex vivo effects of reversible α₄β₁ inhibitors dosed to an experimental animal or humans because compound is washed away during the steps required to isolate the leukocytes of interest from other blood-borne cells. Furthermore, whole blood measurement of α₄β₁-mediated leukocyte adhesion to immobilised α₄β₁ ligands in plastic plates has not been possible because under static conditions red blood cells sediment faster than leukocytes and prevent interactions between the leukocyte α₄β₁ and the immobilised ligand.

Thus there is a need for new methods which overcome the disadvantages of the currently available methods. In particular, new methods are needed which will allow the binding interaction between α₄β₁ and VCAM-1 to be measured in whole blood. Such methods are provided by the methods disclosed in the present invention.

In the present invention, we have developed new methods for measuring the binding interaction between a leukocyte adhesion molecule and a vascular endothelial ligand. A particular advantage of the methods of the invention is that they allow the leukocyte adhesion molecule/vascular endothelial ligand binding interaction to be measured in whole blood. Thus the methods of the invention eliminate the need for isolation of blood leukocytes. It is well known by persons skilled in the art, that the separation and preparative procedures involved in isolation of blood leukocytes alter many leukocyte properties. Traditional methods which require such separation and preparative procedures therefore do not provide an accurate indication of leukocyte adhesion molecule binding to vascular endothelial ligands.

The methods of the invention are especially useful for measuring the effects of compounds which modulate the binding interaction between a leukocyte adhesion molecule and its vascular endothelial ligand in whole blood. The effects of the compounds may be measured in vitro, by spiking blood samples with compounds. Compound effects may also be measured ex vivo, after dosing humans or animals with the compound using any convenient dosing routes.

The methods of the invention may be used to give an indication of the effect of plasma protein binding on the activity of a compound. They also allow pharmacokinetic information on compounds to be linked to activity and duration of action after dosing. In addition, the ability to measure compound activity in whole blood can be used as a surrogate marker for the effectiveness of compounds in human disease so that the effective dose of a compound can be predicted with confidence when undertaking clinical trials.

In the methods of the invention, whole blood is mixed with a mobile solid phase onto which a vascular endothelial ligand of interest is attached, such as via a suitable linker or direct absorption. After incubation, the mobile solid phase and adherent cells are collected and separated from the rest of the blood components, and the binding interaction between the leukocyte adhesion molecule and the vascular endothelial ligand is measured.

The methods of the invention are particularly useful for identifying compounds which modulate the binding interaction between a leukocyte adhesion molecule and a vascular endothelial ligand.

Therefore according to one aspect of the invention, we provide an assay method which comprises:

(i) contacting a leukocyte adhesion molecule in whole blood optionally in the presence of a test compound, with a mobile solid phase coated with a vascular endothelial ligand or a homologue or fragment thereof;

and

(ii) collecting and separating the mobile solid phase and adherent cells from (i)

and

(iii) determining the binding interaction between the leukocyte adhesion molecule and the vascular endothelial ligand

and

(iv) optionally determining whether the test compound modulates the binding interaction between the leukocyte adhesion molecule and the vascular endothelial ligand.

The methods of the invention are useful for measuring the extent of the interaction between a leukocyte adhesion molecule and a vascular endothelial cell ligand in whole blood ex vivo in the absence of test compound, for example to devise optimal assay conditions, to measure inter-subject variability in the interaction, to investigate differences in the interaction because of single nucleotide polymorphisms or to investigate the effect of stimuli that activate leukocyte adhesion.

The methods of the invention are especially useful for determining the effects of compounds on a binding interaction between a leukocyte adhesion molecule and a vascular endothelial ligand in whole blood ex vivo.

Therefore in a preferred embodiment step (i) when performed in the presence of a test compound comprises

(a) administering the test compound to a subject

and

(b) obtaining whole blood from the subject.

According to a further aspect of the invention there is provided an ex vivo whole blood assay method for measuring the binding interaction between a leukocyte adhesion molecule and a vascular endothelial ligand, which comprises:

(i) contacting a leukocyte adhesion molecule in whole blood, optionally in the presence of a test compound, with a mobile solid phase presenting a vascular endothelial ligand or a homologue or fragment thereof;

(ii) collecting and separating the mobile solid phase and adherent cells from (i);

(iii) determining the binding interaction between the leukocyte adhesion molecule and the vascular endothelial ligand; and,

(iv) optionally determining whether the test compound modulates the binding interaction between the leukocyte adhesion molecule and the vascular endothelial ligand.

According to a further aspect of the invention there is provided a method for measuring the ability of a test compound to modulate the binding interaction between a leukocyte adhesion molecule and its vascular endothelial ligand in whole blood comprising:

(a) administering the test compound to a subject;

(b) obtaining whole blood from the subject;

(c) contacting the whole blood with a mobile solid phase presenting a vascular endothelial ligand or a homologue or fragment thereof;

(d) collecting and separating adherent cells bound to the mobile solid phase from other blood components; and

(e) determining the binding interaction between the leukocyte adhesion molecule in the blood and the vascular endothelial ligand.

According to a further aspect of the invention there is provided a method for measuring the ability of a test compound, administered to a subject, to modulate the binding interaction between a leukocyte adhesion molecule and its vascular endothelial ligand in whole blood comprising:

(a) obtaining whole blood from the subject;

(b) contacting the whole blood with a mobile solid phase presenting a vascular endothelial ligand or a homologue or fragment thereof;

(c) collecting and separating adherent cells bound to the mobile solid phase from other blood components; and

(d) determining the binding interaction between the leukocyte adhesion molecule in the blood and the vascular endothelial ligand.

The assay methods of the invention preferably involve measuring or detecting the amount or relative number of cells bound/adhered to the mobile solid phase via the interaction between the leukocyte adhesion molecule on the surface of the white blood cell (leukocyte) and the vascular endothelial ligand form attached to the solid phase. Such cells can be counted manually. Alternatively, indirect measurements such as by measuring the amount of protein bound, measuring the presence of a biomarker present in or on the cell or detecting a label adsorbed onto the cell surface or taken into the cell could also be performed. The person skilled in the art will be able to implement suitable cell detection/measuring methods for use in the invention.

Blood isolated from an individual is usually treated with an anticoagulant to prevent coagulation. The isolated blood is therefore preferably collected into an anticoagulant such as heparin.

The term “leukocyte” is used herein to refer to any type of white blood cell, including lymphocytes, eosinophils, neutrophils, monocytes and macrophages.

The term “leukocyte adhesion molecule” is used herein to refer to an adhesion molecule which is expressed on the surface of a leukocyte, which mediates a binding interaction between the leukocyte and the blood vessel wall.

The term “vascular endothelial ligand” is used herein to refer to an adhesion molecule in the blood vessel wall which undergoes a binding interaction with a leukocyte adhesion molecule. The vascular endothelial ligand may be expressed on the surface of a cell, for example a vascular endothelial cell. Alternatively, the vascular endothelial ligand may be located within the extracellular matrix.

Examples of leukocyte adhesion molecules and their corresponding vascular endothelial ligands, suitable for use in the methods of the present invention, are given in Table 1. (For reviews see Oppenheimer-Marks, N. & Lipsky, P. E., Clin. Imunol. & Immunopathol. 1996, 79, 203-210; Elangbaum, C. S. et al, Vet. Pathol. 1997, 34, 61-73).

TABLE 1

| leukocyte adhesion molecule | corresponding vascular endothelial ligand |
|---|---|
| α₄β₁ integrin | VCAM-1 |
| | fibronectin |
| beta-2 integrin family e.g. | Intercellular Adhesion Molecule (ICAM) |
| LFA-1 (α_{L}β₂, CD11a/CD18) | family e.g. |
| Mac-1 (α_{M}β₂, CD11b/CD18) | ICAM-1 (CD54) |
| α_{X}β₂ (CD11c/CD18) | ICAM-2 (CD102) |
| α_{d}β₂ | ICAM-3 (CD150) |
| α₄β₇ integrin | Mucosal Addressin Cell Adhesion |
| | Molecule (MAdCAM)-1 |
| E-selectin ligands, e.g. | E-selectin (CD62E) |
| cutaneous lymphocyte antigen | |
| (CLA) | |
| sialyl Lewis X (SLeₓ) | |
| P-Selectin Glycoprotein | P-selectin (CD62P) |
| Ligand (PSGL)-1 | |
| L-selectin (CD62L) | Glycosylation-dependent Cell Adhesion |
| | Molecule (GlyCAM)-1 |
| PECAM-1 | Platelet Endothelial Cell Adhesion |
| | Molecule (PECAM)-1 (CD31) |

In a particularly preferred embodiment of the invention, the leukocyte adhesion molecule is the integrin α₄β₁, and the vascular endothelial ligand is VCAM-1.

Any convenient mobile solid phase may be used. The solid support may be of glass, plastics, polymers, polysaccharides, resins, silica or silica-based material, and the like. The term “mobile solid phase” is used herein to refer to solid particulate matter to which a vascular endothelial cell ligand may be attached and which can circulate freely with cells in a blood sample when the blood sample is continually mixed. In a preferred embodiment the solid phase can be collected or separated from other blood components. It will be apparent to a person skilled in the art that various methods are available for collecting or separating the solid phase from blood. The mobile solid phase can be of any shape however, it is most convenient to use bead shaped solid phase. In a preferred aspect of the invention, the solid phase comprises magnetic beads, which includes superparamagentic beads, which may be collected using a magnet. However, other suitable methods include differential centrifugation, flow cytometry, affinity chromatography and the like.

The methods of the invention may be used to measure the binding interaction between a leukocyte adhesion molecule and a vascular endothelial ligand in whole blood from any convenient source. Preferably the whole blood is human or animal blood. Most preferably, the whole blood is human blood.

In some cases the leukocyte adhesion molecules may be in an inactive conformation or may be in low abundance on the cell surface or may be distributed on the cell surface in such a way that interactions with vascular endothelial cell ligands are of low affinity. In such cases the leukocytes may require activation by agents that cause a conformational change in the adhesion molecule or increase its expression or induce clustering in regions of the cell membrane and so increase the affinity and/or avidity of the interaction between the leukocyte adhesion molecule and its vascular endothelial cell ligand. Such changes can be induced by agents that activate certain intracellular signalling pathways in the leukocyte, or interact directly with the adhesion molecule on the leukocyte surface. For example, the leukocyte integrin VLA-4 can be activated by treating leukocytes with some divalent cations or with an activating monoclonal antibody that interact with VLA-4 to induce a conformational change. Chemokines or the anaphylatoxin family of complement components interact with receptors on the leukocyte surface and activate intracellular signalling pathways leading to activation of integrins or increased expression on the leukocyte surface. Activation of leukocyte intracellular protein kinases with phorbol esters can also induce conformational changes, clustering or expression of integrins. Therefore in one embodiment, the whole blood may optionally be treated with appropriate stimuli to activate the binding interaction between a leukocyte adhesion molecule and a vascular endothelial ligand. Examples of appropriate stimuli include a divalent cation such as manganese; an activator of intracellular signalling pathways such as phorbol 12-myristate 13-acetate (PMA); bacterial lipopolysaccharide, leukotriene B₄; the complement component C5a; a member of the chemokine family of chemotactic proteins, for example monocyte chemotactic protein (MCP)-1 or an antibody that binds to integrins causing a change in activation state (Masumoto & Hemler, J. Biol. Chem. 1993, 268, 228-234; Weber C. et al, Proc. Natl. Acad. Sci. USA 1996, 23, 10939-10944; Jakubowski, A. et al, Cell Adhesion & Communication 1995, 3, 131-142; Weber K. S. C. et al, Mol. Biol. Cell 1999, 10, 861-873; Davis et al, J. Immunol. Methods. 2000, 240,125-132).

By the term “homologue” we mean a protein with a substantially similar amino acid sequence to a vascular endothelial ligand protein sequence. The homologue may be a protein from the same species, i.e. a homologous protein family member. Alternatively, the homologue may be a similar protein from a different species such as rat or mouse. Preferably the homologue is a human homologue. Convenient homologues include those which share a sequence similarity of 70% or greater with a vascular endothelial ligand sequence. Preferred sequence similarities include 75% and 80% identity, other preferred sequence similarities include 85% and 90% identity, further preferred sequence similarities include 95% identity.

As used herein the term “vascular endothelial ligand form” refers to each and every full length or variant vascular endothelial ligand that can be used in the invention, i.e. full length, fragments and homologues.

In the case where the vascular endothelial ligand is VCAM-1, convenient homologues include those which share a sequence similarity of 70% or greater with a VCAM-1 sequence as set out in Hession, C. et al, J. Biol. Chem. 1991, 266, 6682-6685 and Osborn, L. et al, Cell 1989, 59, 1203-1211. Preferred sequence similarities include 75% and 80% identity, other preferred sequence similarities include 85% and 90% identity, further preferred sequence similarities include 95% identity.

It is well known that integrins bind to their vascular endothelial cell ligands by recognising short peptide sequences that are presented in the correct conformation by the tertiary structure of the ligand protein. For example, the integrin VLA-4 recognises the Leucine-Aspartic acid-Valine (LDV) tripeptide (in N- to C-terminal order) motif in IIICS domain of fibronectin and the corresponding isoleucine-aspartic acid-serine (IDS) motif found on the first and fourth extracellular domain of VCAM-1 (Komoriya et al, J. Biol. Chem. 1991, 266, 15075-15079; Clements et al, J. Cell Sci. 1994, 107,2127-2135). “Fragments” as used herein, include peptides containing six or more consecutive amino acids of a vascular endothelial ligand that include such a recognition motif presented in the correct conformation so that binding affinity is similar to the parent protein, for example in the case of VLA-4, the 25-amino acid CS-1 peptide or cyclic peptides containing the LDV motif (Haworth et al, Brit. J. Pharmacol. 1999, 126, 1751-1760). In the case where the vascular endothelial ligand is a carbohydrate, for example E-selectin, the term “fragments” includes oligosaccharides containing six or more consecutive monosaccharide units. Fragments of VCAM-1, include peptides containing six or more consecutive amino acids, including the IDS motif, of the VCAM-1 sequence set out in Hession, C. et al, J. Biol. Chem. 1991, 266, 6682-6685 and Osborn, L. et al, Cell 1989, 59, 1203-1211. Preferably the fragments possess the same or essentially the same binding affinity for the leukocyte adhesion molecule as the full length molecule from which they are derived.

In a particularly preferred embodiment, the vascular endothelial ligand is a fragment of VCAM-1 comprising a 7-domain form minus transmembrane and cytoplasmic domains as described in Makarem R. et al, J. Biol. Chem. 1994, 269, 4005-4011. Further preferred fragments of VCAM-1 include a truncated form of VCAM-1 containing at least the first two N-terminal immunoglobulin-like domains (Osborn L. et al, J. Exp. Med. 1992, 176, 99-107); and an alternatively-spliced 6-domain form of VCAM-1 lacking the fourth immunoglobulin-like domain of 7-domain VCAM-1 (Osborn L. et al, Cell 1989, 59, 1203-1211); and a fusion protein comprising at least the first two N-terminal immunoglobulin-like domains of VCAM-1 fused to the Fc region of immunoglobulin G (Jakubowski, A. et al, Cell Adhesion & Communication 1995, 3, 131-142; Vanderslice, P. et al., J. Imunol. 1997, 158, 1710-1718).

Any suitable method that can quantitate the number of adherent leucocytes can be used in this invention. These include for example direct counting of cells using a microscope, measuring protein or DNA concentration or measuring the concentration of a component, i.e. an enzyme or other protein, found in or on or secreted by the leukocytes. In a preferred embodiment the number of adherent leukocytes can be determined using a suitable detectable label,

for example a radioactive label, an antibody or a fluorescent dye. In a particularly preferred embodiment, the marker is a fluorescent dye such as BCECF (2′,7′-bis-(2-carboxyethyl)-5- (and -6)-carboxyfluorescein, acetoxymethyl ester, Molecular Probes B-1150) that is taken up and retained within the cell. The fluorescent signal emitted from cells labelled with BCECF may be readily determined by washing and lysing the cells and measuring using a fluorimeter.

Modulation of the binding interaction between a leukocyte adhesion molecule and a vascular endothelial ligand comprises either stimulation or inhibition. Thus a compound capable of modulating the binding interaction between a leukocyte adhesion molecule and a vascular endothelial ligand is a compound which either stimulates or inhibits the binding interaction between a leukocyte adhesion molecule and a vascular endothelial ligand. The terms “modulator of a leukocyte adhesion molecule/vascular endothelial ligand binding interaction” and “leukocyte adhesion molecule/vascular endothelial ligand modulator” are also used herein to refer to a compound that either stimulates or inhibits the binding interaction between a leukocyte adhesion molecule and a vascular endothelial ligand. The compounds of the invention have utility in the treatment of inflammatory diseases; in general this would arise by inhibition of the binding interaction between a leukocyte adhesion molecule and a vascular endothelial ligand.

Similarly, modulation of the binding interaction between α₄β₁ and VCAM-1 comprises either stimulation or inhibition. Thus a compound capable of modulating the binding interaction between α₄β₁ and VCAM-1 is a compound which either stimulates or inhibits the binding interaction between α₄β₁ and VCAM-1. The terms “modulator of the α₄β₁/VCAM-1 binding interaction” and “α₄β₁/VCAM-1 modulator” are also used herein to refer to a compound that either stimulates or inhibits the α₄β₁/VCAM-1 binding interaction. The compounds of the invention have utility in the treatment of inflammatory diseases; in general this would arise by inhibition of the α₄β₁/VCAM-1 binding interaction.

Compounds which may be tested in the methods of the invention include simple organic molecules, commonly known as “small molecules”, for example those having a molecular weight of less than 2000 Daltons. The methods may also be used to screen compound libraries such as peptide libraries, including synthetic peptide libraries and peptide phage libraries. Other suitable molecules include antibodies, nucleotide sequences and any other molecules which modulate the binding interaction between a leukocyte adhesion molecule and a vascular endothelial ligand.

Once a modulator of the binding interaction between a leukocyte adhesion molecule and a vascular endothelial ligand is identified, then medicinal chemistry techniques can be applied to further refine its properties, for example to enhance efficacy and/or reduce side effects.

The term “determining the effects of a compound ex vivo” as used herein means that a compound is administered to a subject, a blood sample is taken, and the effects of the compound are measured in the blood sample after it has been taken from the subject.

The subject may be of any species, preferably the subject is a human or animal subject.

The compound may be administered to the subject using any suitable route, for example by intravenous, intraperitoneal, subcutaneous or intramuscular injection, or by oral or topical administration. In a preferred embodiment, the compound is administered by oral administration.

The invention will now be illustrated but not limited by reference to the following Examples and Figures.

### FIGURE LEGENDS

FIG. 1

Adhesion of human whole blood cells to VCAM-1-coated magnetic beads is dependent on the interaction between α₄ integrins on the cells and VCAM-1 coating the beads.

FIG. 2

Adhesion of human whole blood cells to VCAM-1-coated magnetic beads is dependent on α₄ β₁ integrin.

FIG. 3

Adhesion of human whole blood cells to VCAM-1 coated magnetic beads is inhibited in a concentration-dependent manner by a small molecule α₄β₁ integrin inhibitor in vitro.

FIG. 4

Adhesion of rat whole blood cells to VCAM-1 coated magnetic beads is inhibited in a concentration-dependent manner by a small molecule α₄β₁ integrin inhibitor in vitro.

FIG. 5

Adhesion of rat whole blood cells to VCAM-1 coated magnetic beads is inhibited ex vivo by continuous intravenous infusion of a small molecule α₄β₁ integrin inhibitor.

FIG. 6

Adhesion of rat whole blood cells to VCAM-1 coated magnetic beads is inhibited ex vivo by bolus intravenous injection of a small molecule α₄β₁ integrin inhibitor.

FIG. 7

Adhesion of rat whole blood cells to VCAM-1 coated magnetic beads is inhibited ex vivo by oral dosing of a small molecule α₄β₁ integrin inhibitor.

FIG. 8

Adhesion of dog whole blood cells to VCAM-1-coated magnetic beads is dependent on the interaction between α₄ integrins on the cells and VCAM-1 coating the beads.

FIG. 9

Adhesion of dog whole blood cells to VCAM-1 coated magnetic beads is inhibited in a concentration-dependent manner by a small molecule α₄β₁ integrin inhibitor in vitro.

FIG. 10

Adhesion of mouse whole blood cells to VCAM-1-coated magnetic beads is α₄ integrin dependent.

### EXAMPLES

### Example 1

### Human VCAM-1 Purification

Recombinant human VCAM-1 (7-domain form minus transmembrane and cytoplasmic domains, Makarem R. et al, J. Biol. Chem. 1994, 269, 4005-4011) was expressed in insect cells using a baculovirus expression system (Sridhar P et al., 1994, J. Biosci., Vol 19, pp603-614) or obtained from R&D Systems Ltd, Abingdon, UK (Catalogue number ADP5). VCAM-1 was purified from insect cells by affinity chromatography as follows. 1G11 monoclonal antibody (RPMS Technology, Hammersmith Hospital, London, UK) was coupled to CNBr activated Sepharose 4B (Pharmacia 17-0430-01) using the manufacturers recommended protocol. A 1G11 affinity column was then packed and equilibrated into 20 mM Tris, 150 mM NaCl, pH 7.4 at 4° C. Insect cell supernatants containing VCAM-1 were then 0.45 μm filtered and loaded on to the 1G11 affinity column. The flow through was monitored at 280 nm. The column was then washed with 20 mM Tris, 150 mM NaCl, pH 7.4 until the trace returned to baseline. The VCAM-1 was then eluted with 0.2 M acetic acid, 150 mM NaCl, pH 2.5. Fractions were immediately neutralised using 2 M Tris HCl, pH 8.0. Fractions containing VCAM-1 were identified by SDS PAGE gel analysis, pooled and dialysed against 20 mM Tris HCl, pH 7.4.

### Example 2

### VCAM-1 Biotinylation

The VCAM-1 was dialysed into 100 mM NaHCO₃, 100 mM NaCl, pH 8.2. To this was added NHS-LC Biotin (Pierce 21335) at a linker to protein ratio of 1:5, and the resulting solution was incubated at room temperature for 1 hour. This was then dialysed into 50 mM Tris HCl, 100 mM NaCl, pH 7.4, both for storage and to remove any unreacted linker.

### Example 3

### Immobilisation Of Biotinylated VCAM-1 To Dynabeads® M-280 Streptavidin

Prior to use, the Dynabeads® M-280 Streptavidin (Dynal (UK) Ltd) were washed twice in Dulbecco's phosphate-buffered saline (PBS, Life Technologies 14040-091) to remove the 0.02% NaN₃ preservative. This was carried out by vortex mixing the Dynabeads® M-280 Streptavidin to obtain a homogeneous suspension and adding 20 mg (2 ml) of Dynabeads® M-280 Streptavidin to a new reaction tube. The tube was then placed in the DynalMPC® (Magnetic Particle Concentrator) for 5 minutes to recover all the beads, and the supernatant was removed by aspiration whilst the tube remained in the Dynal MPC®. The tube was then removed from the Dynal MPC®, 1 ml of Dulbecco's PBS was added, and the tube was inverted gently to resuspend all the beads before being placed back on the Dynal MPC® to repeat the procedure. Finally the supernatant was removed by aspiration to leave a pellet of beads.

Biotinylated VCAM-1 was then added to the washed beads at a concentration of 14.4 μg biotinylated VCAM-1 per mg of beads (800 μl of a stock at 360 μg/ml to 20 mg beads) and the reaction tube placed on a roller mixer for 2 hours at room temperature.

After this incubation period the beads were washed four times as previously described in Dulbecco's PBS containing 0.1% Bovine Serum Albumin (BSA) Fraction V (ICN 810033), and finally resuspended in Dulbecco's PBS containing 0.1% BSA at the original volume (2 ml), and stored at 4° C.

### Example 4

### Whole Blood Collection And Treatment

Whole human or animal blood (e.g. rat, mouse, dog), was collected into sodium heparin (10 Units/ml blood). 1 mM Mn (final concentration) was added to human, dog and mouse blood to induce the activated state of the integrin, but not to rat blood which does not require this step, and then placed on a roller mixer at room temperature for 1 hour before chilling on ice for 15 mins to prevent phagocytosis of the beads.

### Example 5

### Ex Vivo Whole Blood Assay

492.5 μl aliquots of heparinised whole blood from humans or animals dosed with drug or vehicle were dispensed into reaction tubes containing either 7.5 μl biotinylated VCAM-1 beads or uncoated beads, and either with or without 2 μl anti-α₄ intregrin monoclonal antibody to determine maximum inhibition of cell adhesion (e.g. mouse anti-human CD49d, Clone HP2/1, Serotec MCA 697; mouse antirat CD49d, Clone TA-2, Serotec MCA 1383Z; rat anti-mouse CD49d, Clone PS/2, prepared by the method described by Haworth et al, Brit. J. Pharmacol. 1999, 126, 1751-1760 or obtained from Serotec MCA 1230). No anti-dog CD49d antibody is available commercially but anti-human CD49d clone HP2/1 cross-reacts with dog α₄.

### Example 6

### In Vitro Spiked Whole Blood Assay

487.5 μl aliquots of heparinised naive human or animal whole blood were dispensed into reaction tubes containing 7.5 μl biotinylated VCAM beads and either 5 μl of a solution of a compound believed to be an α₄β₁ integrin inhibitor or, as a control, 5 μl of the solvent used to dissolve the compound and either with or without 2 μl anti-α₄ antibody.

### Example 7

### Assay Protocol

Reaction tubes containing ex vivo or in vitro blood samples treated as described above, were rotated on a windmill mixer at 4° C. for 2 hours and then 500 μl chilled PBS was added to each tube and the reaction tubes were placed on a Dynal MPC® magnet for 10 min to recover the beads. The beads were resuspended in 650 μl chilled PBS and aliquoted in triplicate (200 μl aliquots) into a flexible, U-bottomed 96 well plate (Falcon® 353911, Becton Dickinson). The plate was placed on a Dynal MPC® magnet for 5 min and the beads recovered and washed with 200 μl chilled PBS three times. Finally the beads were recovered on the Dynal MPC® magnet and to each well was added 100 μl of 100 μM BCECF-AM (2′,7′-bis-(2-carboxyethyl)-5-(and -6)-carboxyfluorescein, acetoxymethyl ester, Molecular Probes B-1150) and the plate was incubated for 1 hour at room temperature in the dark.

After this time the wash procedure with chilled PBS was carried out three times before the addition of 130 μl per well of 2% Triton X-100 (Sigma T-9284) in distilled water to lyse the cells adhering to the beads. The beads were once again captured by the Dynal MPC® magnet, 100 μl aliquots of the solution from each well was transferred to a clean 96 well flat-bottomed plate and the fluorescence of each well measured using an Fmax fluorimeter (Molecular Devices, Crawley, West Sussex), excitation 485 nm, emission 538 nm.

### Example 8

### Adhesion Of Human Whole Blood Cells To VCAM-1-coated Magnetic Beads Is Dependent On The Interaction Between α₄ Integrins On The Cells And VCAM-1 Coating The Beads

Human blood (20 ml) was collected into sodium heparin (10 units/ml) and divided into 10 ml aliquots. Manganese chloride (1 mM final concentration) was added to one aliquot and the blood was incubated on a roller mixer at room temperature for 1 h. After cooling on ice for 15 min, aliquots of blood (490.5 μl) were dispensed into polypropylene microcentrifuge tubes containing 7.5 μl VCAM-1 coated magnetic beads or uncoated beads and 2 μl mouse anti-human α₄, clone HP2/1 (4 μg/ml) or isotype control, mouse IgG1 (4 μg/ml) or PBS.

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no antibody.

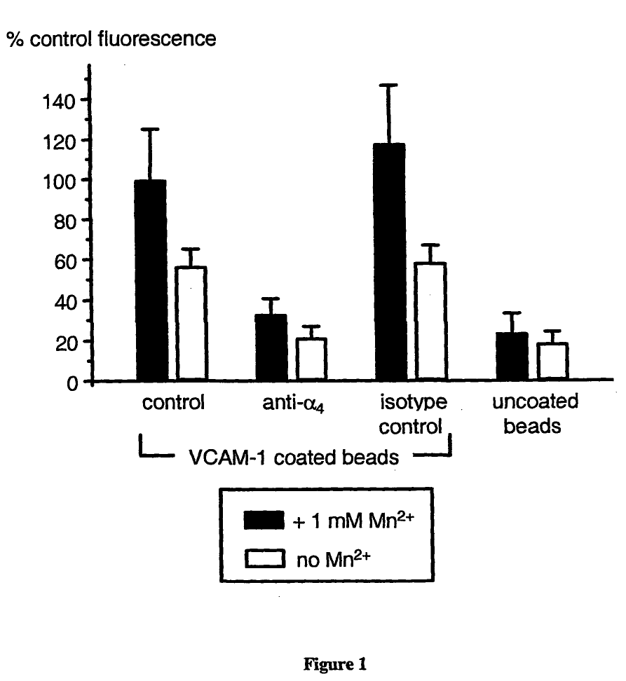

Incubation with Mn²⁺ induced approximately 2-fold greater whole blood cell adhesion in control samples. Cell adhesion with or without Mn²⁺ was inhibited by a monoclonal antibody to the α₄ integin subunit while the isotype control had no effect. There was little cell adhesion to beads not coated with VCAM-1 with or without Mn₂₊. These results indicate that the adhesion of human whole blood cells to VCAM-1 coated magnetic beads is largely dependent on the interaction between α₄ integrins on the cell surface and VCAM-1 coating the beads. (FIG. 1)

### Example 9

### Adhesion Of Human Whole Blood Cells To VCAM-1-coated Magnetic Beads Is Dependent On α₄β₁ Integrin

Human blood (20 ml) was collected into sodium heparin (10 units/ml) and incubated on a roller mixer with 1 mM manganese chloride at room temperature for 1 h. After cooling on ice for 15 min, aliquots of blood (490.5 μl) were dispensed into polypropylene microcentrifuge tubes containing 7.5 μl VCAM-1 coated magnetic beads and 2 μl mouse anti-human α₄, clone HP2/1(4 μg/ml) or mouse anti-human β₁, clone 3S3 (4 μg/ml) or isotype control, mouse IgG1 (4 μg/ml) or PBS.

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no antibody.

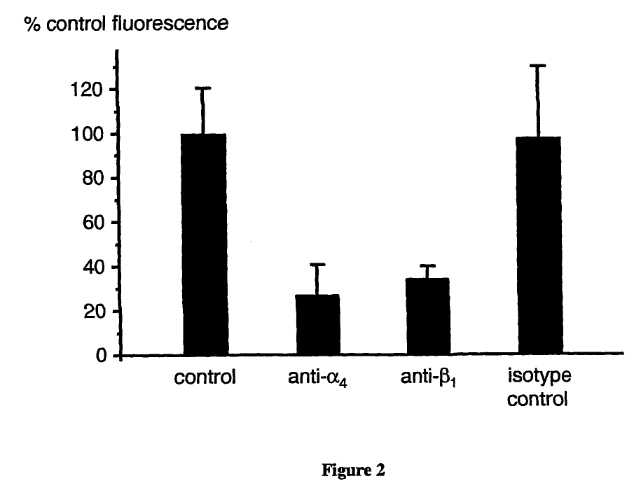

Antibodies to the α₄ and β₁ integin subunits inhibited human whole blood cell adhesion while the isotype control had no effect, showing that the majority of the adhesion is dependent on the α₄β₁ integrin (FIG. 2).

### Example 10

### Adhesion Of Human Whole Blood Cells To VCAM-1 Coated Magnetic Beads Is Inhibited In A Concentration-dependent Manner By A Small Molecule α₄β₁ Integrin Inhibitor In Vitro

Human blood (20 ml) was collected into sodium heparin (10 units/ml) and incubated on a roller mixer with 1 mM manganese chloride at room temperature for 1 h. After cooling on ice for 15 min, aliquots of blood (487.5 μl) were dispensed into polypropylene microcentrifuge tubes containing 7.5 μl VCAM-1 coated magnetic beads and 5 μl of dilutions of a small molecule α₄β₁ integrin inhibitor (0.01 to 10 μM final concentration) or PBS or 2 μl mouse anti-human α₄ monoclonal antibody, clone HP2/1 (4 μg/ml).

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluoresence in control samples containing no inhibitor.

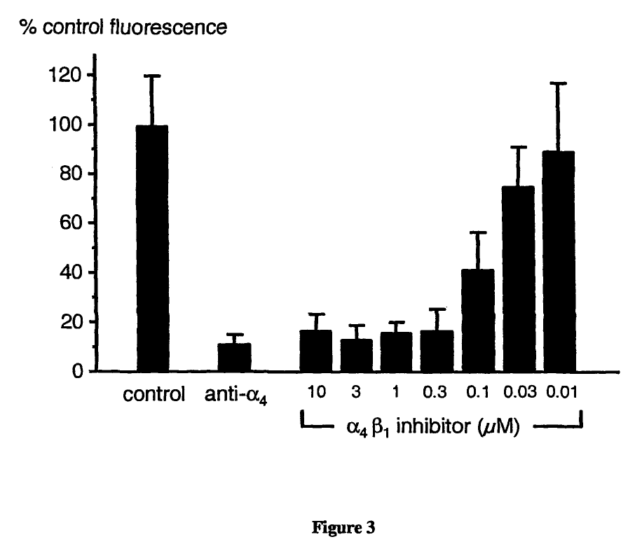

The small molecule α₄β₁ integrin inhibitor inhibited human whole blood cell adhesion to the VCAM-1 coated magnetic beads in a concentration-dependent manner. Maximum inhibition was equivalent to that obtained with the anti-α₄ monoclonal antibody. The concentration of the small molecule inhibitor required to inhibit human whole blood cell adhesion by 50% (IC₅₀) was estimated to be 0.06 μM. (FIG. 3).

### Example 11

### Adhesion Of Rat Whole Blood Cells To VCAM-1 Coated Magnetic Beads Is Inhibited In A Concentration-dependent Manner By A Small Molecule α₄β₁ Integrin Inhibitor In Vitro

Pooled, heparinised whole blood from AP strain rats (20 ml) was incubated on a roller mixer at room temperature for 1 h. After cooling on ice for 15 min, aliquots of blood (487.5 μl) were dispensed into polypropylene microcentrifuge tubes containing 7.5 μl VCAM-1 coated magnetic beads and 5 μl of dilutions of a small molecule α₄β₁ integrin inhibitor (0.03 to 10 μM final concentration) or PBS or 2 μl mouse anti-rat α₄ monoclonal antibody, clone TA-2 (4 μg/ml).

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no inhibitor.

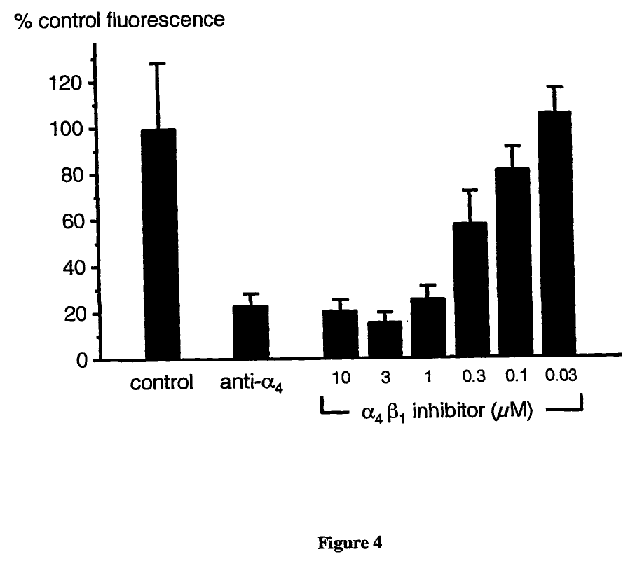

The small molecule α₄β₁ integrin inhibitor dose-dependently inhibited rat whole blood cell adhesion to the VCAM-1 coated magnetic beads. Maximum inhibition was equivalent to that obtained with the anti-α₄ monoclonal antibody. The concentration of the small molecule inhibitor required to inhibit rat whole blood cell adhesion by 50% (IC₅₀) was estimated to be 0.3 μM. (FIG. 4).

### Example 12

### Adhesion Of Rat Whole Blood Cells To VCAM-1 Coated Magnetic Beads Is Inhibited Ex Vivo By Continuous Intravenous Infusion Of A Small Molecule α₄β₁ Integrin Inhibitor

Groups of five rats were dosed with a small molecule α₄β₁ integrin inhibitor (10 mg/kg/day) or with saline (240 μl/day) by continuous subcutaneous infusion from osmotic mini-pumps. After 48 h, when plasma levels of the α₄ β₁ integrin inhibitor had reached steady state, the rats were killed and 2 ml blood samples were collected from each rat into heparin (10 units/ml). Each blood sample was incubated on a roller mixer at room temperature for 0.5 h. After cooling on ice for 15 min, two aliquots of blood from each rat (492.5 μl) were dispensed into polypropylene microcentrifuge tubes containing 7.5 μl VCAM-1 coated magnetic beads with or without 2 μl of a mouse anti-rat α₄ monoclonal antibody, clone TA-2 (4 μg/ml).

The tubes were rotated on a windmill mixer for 2 h at 4° C. before addition of 500 μl ice cold Dulbecco's phosphate-buffered saline containing 0.1% BSA (PBS) and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the maximum fluorescence in samples from rats infused with saline.

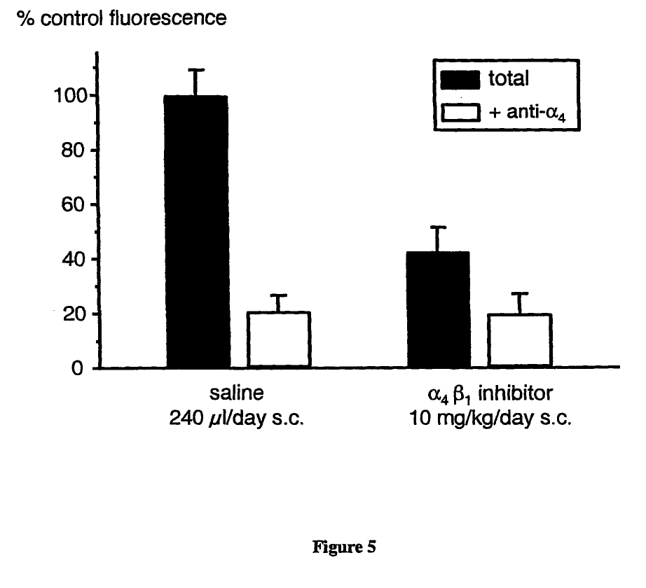

Continuous infusion of the small molecule α₄β₁ integrin inhibitor inhibited α₄ integrin-dependent whole blood cell adhesion ex vivo (the difference between the total fluorescence and the fluorescence in the presence of the anti-rat α₄ antibody) by 71% (P<0.001, Student's t-test). (FIG. 5).

### Example 13

### Adhesion Of Rat Whole Blood Cells To VCAM-1 Coated Magnetic Beads Is Inhibited Ex Vivo By Bolus Intravenous Injection Of A Small Molecule α₄β₁ Integrin Inhibitor

Groups of three rats were dosed with a small molecule α₄β₁ integrin inhibitor (10 mg/kg) or with vehicle (5 ml/kg) by bolus intravenous injection. At 10, 30 or 120 min post-injection, groups of rats were killed and 2 ml blood samples were collected from each rat into heparin (10 units/ml). Each blood sample was incubated on a roller mixer at room temperature for 0.5 h. After cooling on ice for 15 min, two aliquots of blood from each rat (492.5 μl) were dispensed into polypropylene microcentrifuge tubes containing 7.5 μl VCAM-1 coated magnetic beads either with or without 2 μl of a mouse anti-rat α₄ monoclonal antibody, clone TA-2 (4 μg/ml).

The tubes were rotated on a windmill mixer for 2 h at 4° C. before addition of 500 μl ice cold Dulbecco's phosphate-buffered saline containing 0.1% BSA (PBS) and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. Finally the beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. At each time post-dose, results were expressed as a percentage of the maximum fluorescence in samples from rats injected with vehicle. The concentration of the α₄β₁ integrin inhibitor in an aliquot of each plasma sample was measured by liquid chromatography-mass spectrometry.

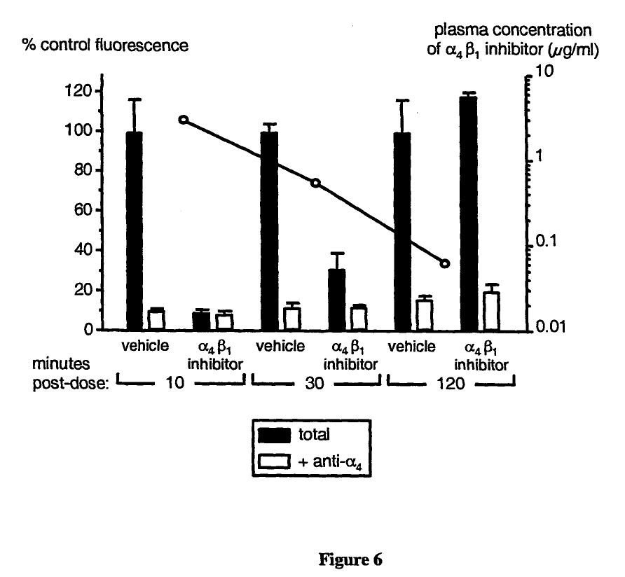

At 10 min post-dose, the small molecule α₄β₁ integrin inhibitor reduced α₄ integrin-dependent whole blood cell adhesion ex vivo (the difference between the total fluorescence and the fluorescence in the presence of the anti-rat α₄ antibody) by 99% (P<0.001, Student's t-test). At 30 min post-dose the inhibition was 78% (P<0.001) while at 120 min post-dose there was no inhibition of whole blood cell adhesion. The loss of inhibition with time post-injection was consistent with the decline of plasma levels of the inhibitor and the concentration of inhibitor required to inhibit rat whole blood cell adhesion in vitro by 50% (0.2 μg/ml). (FIG. 6).

### Example 14

### Adhesion Of Rat Whole Blood Cells To VCAM-1 Coated Magnetic Beads Is Inhibited Ex Vivo By Oral Dosing Of A Small Molecule α₄β₁ Integrin Inhibitor

Groups of four rats were dosed orally with a small molecule α₄β₁ integrin inhibitor (20 mg/kg) or with vehicle (5 ml/kg). At 1 h post-dose, blood samples were collected from the tail vein of each rat into heparin (10 units/ml). At 2 h post-dose, the rats were killed and 2 ml blood samples were collected from each rat into heparin. Each blood sample was incubated on a roller mixer at room temperature for 0.5 h. After cooling on ice for 15 min, two aliquots of blood from each rat (492.5 μl) were dispensed into polypropylene microcentrifuge tubes containing 7.5 μl VCAM-1 coated magnetic beads either with or without 2 μl of a mouse anti-rat α₄ monoclonal antibody, clone TA-2 (4 μg/ml).

The tubes were rotated on a windmill mixer for 2 h at 4° C. before addition of 500 μl ice cold Dulbecco's phosphate-buffered saline containing 0.1% BSA (PBS) and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. At each time post-dose, results were expressed as a percentage of the maximum fluorescence in samples from rats dosed with vehicle. The concentration of the α₄β₁ integrin inhibitor in an aliquot of each plasma sample was measured by liquid chromatography-mass spectrometry.

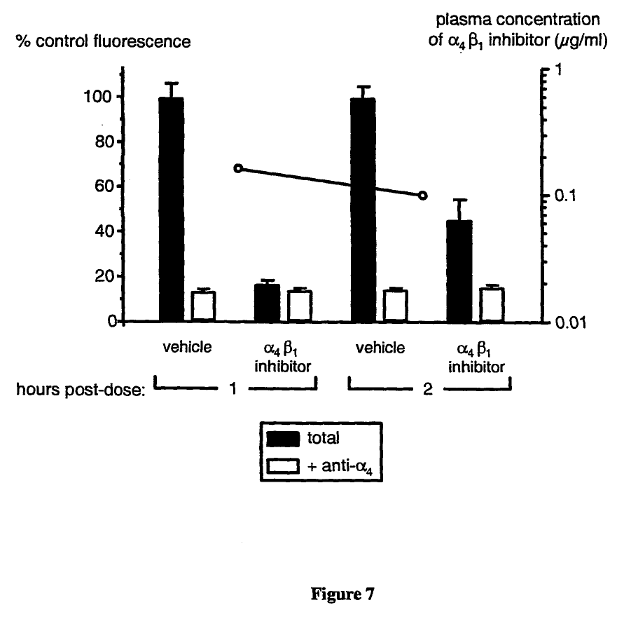

At 1 h post-dose, the small molecule α₄β₁ integrin inhibitor reduced α₄ integrin-dependent whole blood cell adhesion ex vivo (the difference between the total fluorescence and the fluorescence in the presence of the anti-rat N antibody) by 97% (P<0.001, Student's t-test). At 2 h post-dose, the inhibition was 65% (P<0.001). The loss of inhibition with time post-injection was consistent with the decline of plasma levels of the inhibitor and the concentration of inhibitor required to inhibit rat whole blood cell adhesion in vitro by 50% (0.03 μg/ml). (FIG. 7).

### Example 15

### Adhesion Of Dog Whole Blood Cells To VCAM-1-coated Magnetic Beads Is Dependent On The Interaction Between α₄ Integrins On The Cells And VCAM-1 Coating The Beads

Dog blood (20 ml) was collected into sodium heparin (10 units/ml). Manganese chloride (1 mM final concentration) was added and the blood was incubated on a roller mixer at room temperature for 1 h. After cooling on ice for 15 min, aliquots of blood (490.5 μl) were dispensed into polypropylene microcentrifuge tubes containing 7.5 μl VCAM-1 coated magnetic beads and 2 μl mouse anti-human α₄, clone HP2/1 (4 μg/ml) or isotype control, mouse IgG1 (4 μg/ml) or PBS.

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no antibody.

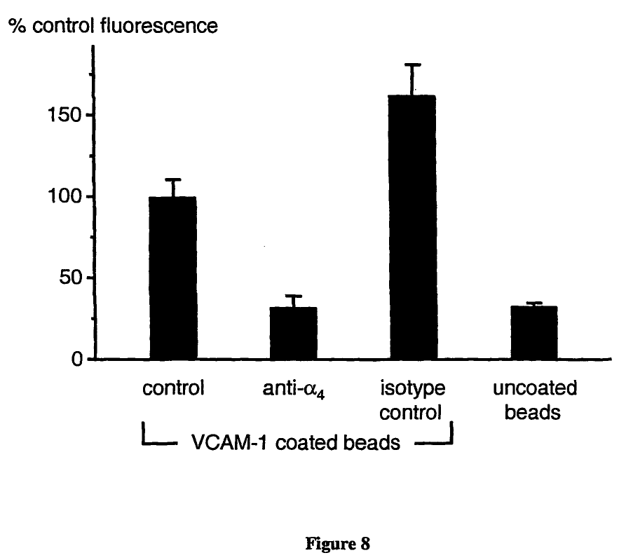

Dog whole blood cell adhesion was inhibited by a monoclonal antibody to the human α₄ integrin subunit (that cross-reacts with dog α₄) while the isotype control had no effect. There was little cell adhesion to beads not coated with VCAM-1. These results indicate that the adhesion of dog whole blood cells to VCAM-1 coated magnetic beads is dependent on the interaction between α₄ integrins on the cell surface and VCAM-1 coating the beads. (FIG. 8).

### Example 16

### Adhesion Of Dog Whole Blood Cells To VCAM-1 Coated Magnetic Beads Is Inhibited In A Concentration-dependent Manner By A Small Molecule α₄β₁ Integrin Inhibitor In Vitro

Dog blood (20 ml) was collected into sodium heparin (10 units/ml) and incubated on a roller mixer with 1 mM manganese chloride at room temperature for 1 h. After cooling on ice for 15 min, aliquots of blood (487.5 μl) were dispensed into polypropylene microcentrifuge tubes containing 7.5 μl VCAM-1 coated magnetic beads and 5 μl of dilutions of a small molecule α₄β₁ integrin inhibitor (0.03 to 30 μM final concentration) or PBS or 2 μl mouse anti-human α₄ monoclonal antibody, clone HP2/1 (4 μg/ml).

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. Finally the beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no inhibitor.

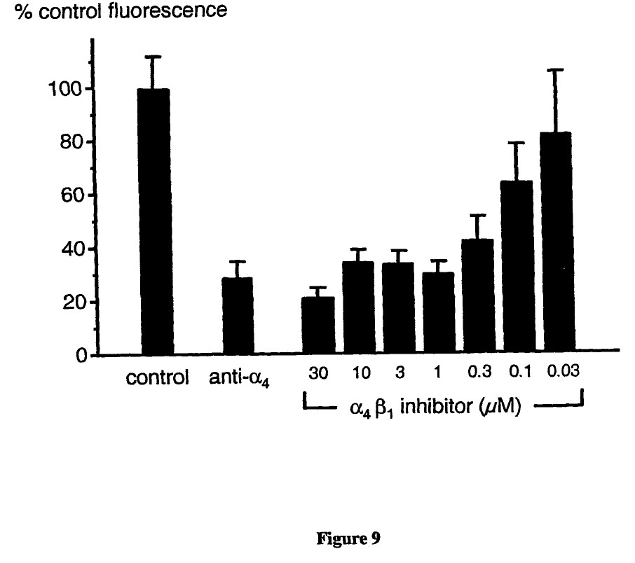

The small molecule α₄β₁ integrin inhibitor inhibited dog whole blood cell adhesion to the VCAM-1 coated magnetic beads in a concentration-dependent manner. Maximum inhibition was equivalent to that obtained with the anti-α₄ monoclonal antibody. The concentration of the small molecule inhibitor required to inhibit dog whole blood cell adhesion by 50% (IC₅₀) was estimated to be 0.1 μM. (FIG. 9).

### Example 17

### Adhesion Of Mouse Whole Blood Cells To VCAM-1-coated Magnetic Beads Is α₄ Integrin Dependent

Mouse blood was collected into sodium heparin (10 units/ml) and pooled. Manganese chloride (1 μM final concentration) was added to 2.5 ml aliquots and the blood was incubated on a roller mixer at room temperature for 0.5 h. After cooling on ice for 15 min, aliquots of blood (492.5 μl) were dispensed into polypropylene microcentrifuge tubes containing 7.5 μl VCAM-1 coated magnetic beads and 2 μl rat anti-mouse α₄, clone PS/2 (4 μg/ml) or isotype control, rat IgG2bκ (4 μg/ml) or PBS. Alternatively 487.5 μl blood were dispensed into tubes containing 7.5 μl VCAM-1 coated magnetic beads and 5 μl of a small molecule α₄β₁ integrin inhibitor (3 μM final concentration).

The tubes were rotated on a windmill mixer for 2 h at 4° C. before addition of 500 μl ice cold PBS and placing on a Dynal MPC® magnet for 10 min to recover the beads. The beads were resuspended in 650 μl chilled PBS and aliquoted in triplicate (200 μl aliquots) into a 96 well, flexible, U-bottomed plate. The plate was placed on a Dynal MPC® magnet for 5 min and the beads recovered and washed with 200 μl chilled PBS three times. The beads were recovered on the Dynal MPC® magnet and to each well was added 100 μl of 100 μM BCECF-AM and the plate incubated for 1 h at room temperature in the dark. After this time, the washing procedure with chilled PBS was carried out three times before the addition of 130 μl per well of 2% Triton X-100 in distilled water to lyse the cells adhering to the beads. The beads were once again captured by the Dynal MPC® magnet, before transferring 100 μl aliquots of the solution from each well to a clean 96 well flat-bottomed plate. The fluorescence of each well was measured using a fluorimeter, excitation 485 nm, emission 538 nm. Results were expressed as a percentage of the fluorescence in control samples containing no antibody.

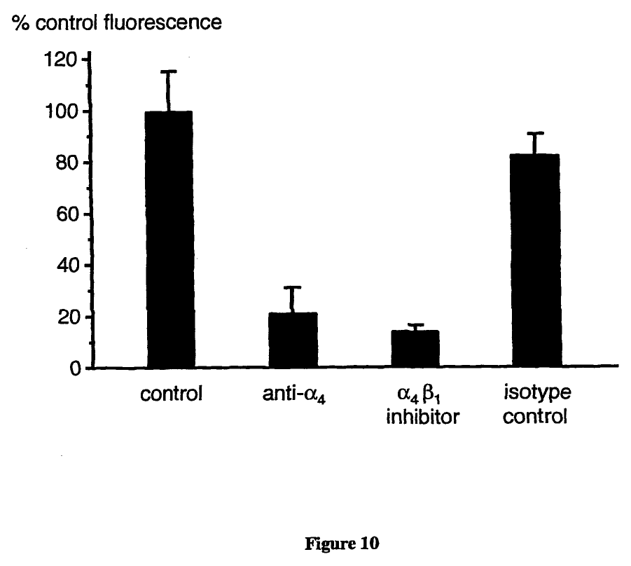

Mouse whole blood cell adhesion was inhibited by a monoclonal antibody to the mouse α₄ integrin subunit while the isotype control had no effect. A small molecule α₄β₁ integrin inhibitor also inhibited mouse whole blood cell adhesion in vitro. These results indicate that the adhesion of mouse whole blood cells to VCAM-1 coated magnetic beads is largely α₄ integrin dependent. (FIG. 10).

1. An ex vivo whole blood assay method for measuring the binding interaction between a leukocyte adhesion molecule and a vascular endothelial ligand, which comprises:
(i) contacting a leukocyte adhesion molecule in whole blood, optionally in the presence of a test compound, with a mobile solid phase presenting a vascular endothelial ligand or a homologue or fragment thereof;
(ii) collecting and separating the mobile solid phase and adherent cells from (i);
(iii) determining the binding interaction between the leukocyte adhesion molecule and the vascular endothelial ligand; and,
(iv) optionally determining whether the test compound modulates the binding interaction between the leukocyte adhesion molecule and the vascular endothelial ligand.

2. A method as claimed in claim 1, wherein step (i) when performed in the presence of a test compound comprises:
(a) administering the test compound to a subject; and,
(b) obtaining whole blood from the subject.

3. A method for measuring the ability of a test compound to modulate the binding interaction between a leukocyte adhesion molecule and its vascular endothelial ligand in whole blood comprising:
(f) administering the test compound to a subject;
(g) obtaining whole blood from the subject;
(h) contacting the whole blood with a mobile solid phase presenting a vascular endothelial ligand or a homologue or fragment thereof;
(i) collecting and separating adherent cells bound to the mobile solid phase from other blood components; and
(j) determining the binding interaction between the leukocyte adhesion molecule in the blood and the vascular endothelial ligand.

4. A method for measuring the ability of a test compound, administered to a subject, to modulate the binding interaction between a leukocyte adhesion molecule and its vascular endothelial ligand in whole blood comprising:
(e) obtaining whole blood from the subject;
(f) contacting the whole blood with a mobile solid phase presenting a vascular endothelial ligand or a homologue or fragment thereof;
(g) collecting and separating adherent cells bound to the mobile solid phase from other blood components; and
(h) determining the binding interaction between the leukocyte adhesion molecule in the blood and the vascular endothelial ligand.

5. A method as claimed in any of the preceding claims wherein the amount or relative number of adherent cells are measured.

6. A method as claimed in claim 4 wherein the amount or relative number of adherent cells are measured by labelling the cells with a marker, such as a radioactive marker, an antibody or a fluorescent dye.

7. A method as claimed in claim 6 wherein the marker is the fluorescent dye: BCECF (2′,7′-bis-(2-carboxyethyl)-5-(and -6)-carboxyfluorescein, acetoxymethyl ester).

8. A method as claimed in any of the preceding claims wherein the vascular endothelial ligand or a homologue or fragment thereof, is selected from the group consisting of: VCAM-1, fibronectin, Intercellular Adhesion Molecule-1(ICAM-1; CD54), ICAM-2 (CD102), ICAM-3 (CD150), Mucosal Addressin Cell Adhesion Molecule (MAdCAM)-1, E-selectin (CD62E), P-selectin (CD62P), Glycosylation-dependent Cell Adhesion Molecule (GlyCAM)-1 and Platelet Endothelial Cell Adhesion Molecule (PECAM)-1 (CD31).

9. A method as claimed in any of the preceding claims wherein the vascular endothelial ligand is VCAM-1, a homologue thereof or fragment of either.

10. A method as claimed in any of the preceding claims wherein the mobile solid phase is or comprises magnetic beads.

11. A method as claimed in any of the preceding claims wherein the whole blood is treated with a stimulus to activate the binding interaction between a leukocyte adhesion molecule and a vascular endothelial ligand.

12. A method as claimed in claim 11 wherein the stimulus is selected from the group consisting of: manganese; an activator of intracellular signalling pathways, such as phorbol 12-myristate 13-acetate (PMA) or bacterial lipopolysaccharide;; a member of the chemokine family of chemotactic proteins, for example monocyte chemotactic protein (MCP)-1; an anaphylatoxin, such as C5a; other chemotactic agents such as leukotriene B₄; and, an antibody that binds to the leukocyte adhesion molecule causing a change in activation state.

13. A method as claimed in claim 11 or 12, wherein the stimulus is manganese.
